(19) United States
(12) Patent Application Publication
(10) Pub. No.: US 2022/0354437 A1
(43) Pub. Date: Nov. 10, 2022

(54) METHOD FOR RISK ASSESSMENT OF NEUROLOGICAL DISORDER AND ELECTRONIC DEVICE USING THE SAME

(71) Applicant: Hung-Yi Hsu, Taichung City (TW)

(72) Inventors: Hung-Yi Hsu, Taichung City (TW); Wen-Yung Sheng, Taipei City (TW); Ke Han, Shenzhen (CN)

(73) Assignee: Hung-Yi Hsu, Taichung City (TW)

(21) Appl. No.: 17/736,056

(22) Filed: May 3, 2022

(30) Foreign Application Priority Data
May 6, 2021 (TW) 110116312

(51) Int. Cl. A61B 5/00; A61B 8/06; A61B 5/026; G16H 50/30

(52) U.S. Cl. CPC A61B 5/7275; A61B 8/06; A61B 5/026; G16H 50/30

(57) ABSTRACT

A method for risk assessment of neurological disorder and an electronic device using the same method are provided. The method for risk assessment includes the following. A blood flow signal is obtained. Signal decomposition is performed on the blood flow signal to generate a first signal and a second signal. The first signal is de-modulated to generate a modulation signal. A correlation signal is generated according to the modulation signal and the second signal. A statistical parameter is generated according to the correlation signal. Whether to output a warning message is determined according to the statistical parameter.

### CROSS-REFERENCE TO RELATED APPLICATION

This application claims the priority benefit of Taiwan application serial no. 110116312, filed on May 6, 2021. The entirety of the above-mentioned patent application is hereby incorporated by reference herein and made a part of this specification.

### BACKGROUND

### Technical Field

The disclosure relates to a method for risk assessment of neurological disorders and an electronic device using the same method.

### Description of Related Art

Adequate cerebral blood supply and metabolism are crucial to maintain normal neurological function. The cerebrovascular regulation involved complicated mechanisms and has an important role in various neurological disorders (for example, abnormal heartbeat, dizziness, blurred vision, abnormal body temperature control, instability of blood pressure, dysphagia, sleep disturbance, memory loss, degraded language and space ability, reduced concentration, declined cognitive performance, or psychomotor symptoms such as irritability or depression). Early detection of neurological disorders and timing of treatment are vital to a patient's recovery. Early diagnosis and evaluation of symptoms involving major neurocognitive disorder caused by degenerative neurocognitive function is even more difficult in clinical practice. The risk of neurological disorders can be evaluated by assessment of dynamic changes of cerebrovascular regulation.

### SUMMARY

The disclosure provides a method for risk assessment of neurological disorder, which assesses whether a subject is at risk of neurological disorder, and an electronic device using the same method.

An electronic device for risk assessment of neurological disorder of the disclosure includes a processor, a storage medium, and a transmitter. The storage medium stores multiple modules. The processor is coupled to the storage medium and the transformer, and accesses and performs multiple modules. The modules include a data collection module and a calculation module. The data collection module obtains a blood flow signal through the transmitter. The calculation module performs signal decomposition on the blood flow signal to generate a first signal and a second signal, de-modulates the first signal to generate a modulation signal, generates a correlation signal according to the modulation signal and the second signal, generates a statistical parameter according to the correlation signal, and determines whether to output a warning message through the transmitter according to the statistical parameter.

In an embodiment of the disclosure, the blood flow signal as described above is a cerebral blood flow velocity signal, the first signal is a blood pulse signal, and the second signal is a trend signal.

In an embodiment of the disclosure, the calculation module as described above performs the signal decomposition according to one of the following: peak-valley interpolation, empirical mode decomposition, and de-trend fluctuation analysis.

In an embodiment of the disclosure, the calculation module as described above performs the peak-valley interpolation on the blood flow signal to generate the first signal, and subtracts the first signal from the blood flow signal to generate the second signal.

In an embodiment of the disclosure, the calculation module as described above performs the empirical mode decomposition on the blood flow signal to generate the first signal and the second signal, and the first signal is an intrinsic mode signal, and the second signal is a residue signal.

In an embodiment of the disclosure, the calculation module as described above performs the de-trend fluctuation analysis on the blood flow signal to generate the first signal, and subtracts the first signal from the blood flow signal to generate the second signal.

In an embodiment of the disclosure, the statistical parameter as described above at least includes one of the following: a mean, a standard deviation, an interquartile range, and a coefficient of variation.

In an embodiment of the disclosure, the modulation signal as described above corresponds to amplitude modulation.

In an embodiment of the disclosure, the calculation module as described above determines whether to output a warning message according to the correlation signal and the statistical parameter.

A method for risk assessment of neurological disorder of the disclosure includes the following. A blood flow signal is obtained. Signal decomposition is performed on the blood flow signal to generate a first signal and a second signal. The first signal is de-modulated to generate a modulation signal. A correlation signal is generated according to the modulation signal and the second signal. A statistical parameter is generated according to the correlation signal. Whether to output a warning message is determined according to the statistical parameter.

Based on the above, the electronic device of the disclosure determines whether a subject is at risk of neurological disorder according to the subject's blood flow signal. If the electronic device determines that the subject is at risk of neurological disorder, the electronic device outputs a warning message to notify a relevant person.

### BRIEF DESCRIPTION OF THE DRAWINGS

FIG. 1 illustrates a schematic diagram of an electronic device for risk assessment of neurological disorder according to an embodiment of the disclosure.

FIG. 2 illustrates a schematic diagram of a method for risk assessment of neurological disorder according to an embodiment of the disclosure.

FIG. 3 illustrates a flow chart of a method for risk assessment of neurological disorder according to an embodiment of the disclosure.

### DESCRIPTION OF THE EMBODIMENTS

In order for the content of the disclosure to be easier to understand, embodiments are described below as examples of how the disclosure may actually be implemented. In addition, wherever possible, elements/components/steps with the same symbol in the drawings and embodiments represent same or like components.

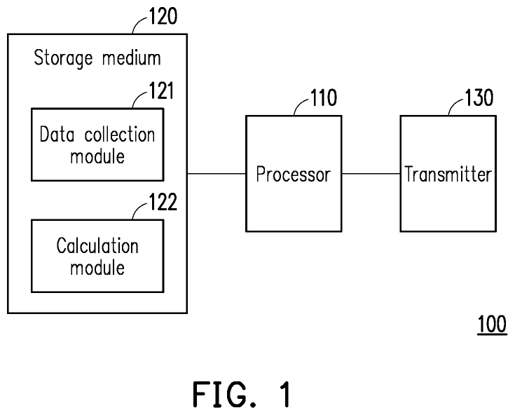

FIG. 1 illustrates a schematic diagram of an electronic device for risk assessment of neurological disorder according to an embodiment of the disclosure. An electronic device 100 may include a processor 110, a storage medium 120, and a transmitter 130.

The processor 110 is, for example, a central processing unit (CPU), or other programmable general-purpose or special-purpose elements, for example, a micro control unit (MCU), a microprocessor, a digital signal processor (DSP), a programmable controller, an application specific integrated circuit (ASIC), a graphics processing unit (GPU), an image signal processor (ISP), an image processing unit (IPU), an arithmetic logic unit (ALU), a complex programmable logic device (CPLD), a field programmable gate array (FPGA), or other like elements or a combination of the above elements. The processor 110 may be coupled to the storage medium 120 and the transmitter 130, and may access and perform a plurality of modules and various applications stored in the storage medium 120.

The storage medium 120 is, for example, any type of fixed or removable element, for example, a random access memory (RAM), a read-only memory (ROM), a flash memory, a hard disk drive (HDD), a solid state drive (SSD), or a like element or a combination of the above elements, and is configured to store a plurality of modules and various applications that may be performed by the processor 110. In this embodiment, the storage medium 120 may store a plurality of modules including a data collection module 121 and a calculation module 122, and functions thereof will be explained later.

The transceiver 130 transmits and receives signals in a wireless or wired manner. The transceiver 130 may further perform operations such as low noise amplification, impedance matching, frequency mixing, up or down frequency conversion, filtering, amplification, and like operations.

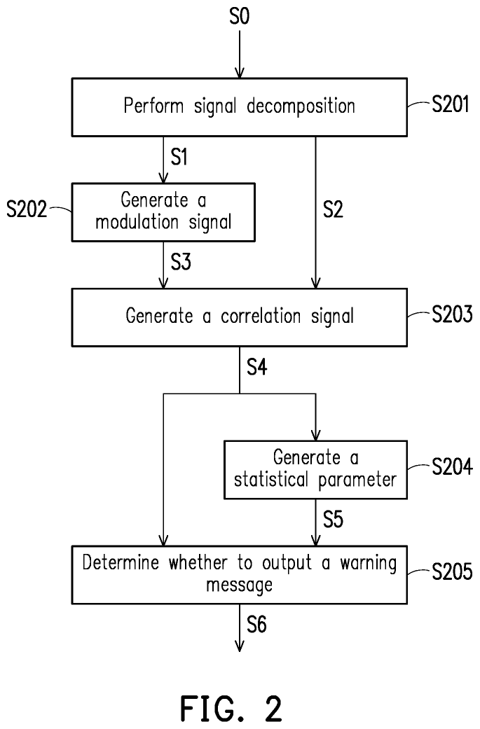

FIG. 2 illustrates a schematic diagram of a method for risk assessment of neurological disorder according to an embodiment of the disclosure, and the method for risk assessment may be implemented by the electronic device 100 shown in FIG. 1. First, the data collection module 121 may obtain a blood flow signal S0 of a subject through the transmitter 130. The blood flow signal S0 may be a time-varying signal. The blood flow signal S0 is, for example, a cerebral blood flow velocity (CBFV) signal. For example, the data collection module 121 may communicate with an external apparatus for measuring the blood flow signal through the transmitter 130, thereby obtaining the blood flow signal S0 from the external apparatus.

In step S201, the calculation module 122 may perform signal decomposition on the blood flow signal S0 to generate a signal S1 and a signal S2. The signal S1 is, for example, a blood pulse signal, and the signal S2 is, for example, a trend signal. A signal decomposition algorithm may be configured according to needs, and the disclosure is not limited thereto. The signal S1 or the signal S2 may be a time-varying signal.

In an embodiment, the calculation module 122 may obtain a trend signal of the blood flow signal S0 through peak-valley interpolation, and perform signal decomposition on the blood flow signal S0 by subtracting the trend signal. Specifically, the calculation module 122 may perform peak-valley interpolation on the blood flow signal S0 to generate the signal S1 (that is, the trend signal of the blood flow signal S0). Next, the calculation module 122 may subtract the signal S1 from the blood flow signal S0 to generate the signal S2.

In an embodiment, the calculation module 122 may perform signal decomposition on the blood flow signal S0 through an empirical mode decomposition (EMD) method. Specifically, the calculation module 122 may perform the empirical mode decomposition method on the blood flow signal S0 to generate the signal S1 and the signal S2. The signal S1 is an intrinsic mode signal (for example, IMF1) corresponding to the empirical mode decomposition method, and the signal S2 is a residue signal corresponding to the empirical mode decomposition method.

In an embodiment, by using a de-trend fluctuation analysis (DFA), the calculation module 122 may use interpolation at different time scales to obtain a trend signal of the blood flow signal S0 at different time scales, and perform signal decomposition on the blood flow signal S0 by subtracting the trend signal. Specifically, the calculation module 122 may perform a de-trend fluctuation analysis on the blood flow signal S0 to generate the signal S1 (that is, the trend signal of the blood flow signal S0 at different time scales). Next, the calculation module 122 may subtract the signal S1 from the blood flow signal S0 to generate the signal S2.

In step S202, the calculation module 122 may de-modulate the modulation signal S1 to generate a modulation signal S3. Specifically, the calculation module 122 may perform amplitude modulation (AM) on the signal S1 to generate the modulation signal S3. The modulation signal S3 may be a time-varying signal.

In step S203, the calculation module 122 may generate a correlation signal S4 according to the modulation signal S3 and the signal S2. The correlation signal S4 may be a time-varying signal, and may include one or more correlation coefficients, respectively corresponding to different time periods. The correlation coefficient is, for example, a Pearson product-moment correlation coefficient, but the disclosure is not limited thereto.

In step S204, the calculation module 122 may generate a statistical parameter S5 according to the correlation signal S4. The statistical parameter S5 is, for example, a mean, a standard deviation, an interquartile range (IQR), or a coefficient of variation (CV) of the correlation signal S4, but the disclosure is not limited thereto.

In step S205, the calculation module 122 may determine whether to output a warning message S6 through the transmitter 130 according to the statistical parameter S5. For example, assuming that the statistical parameter S5 is a coefficient of variation, the calculation module 122 may determine to output a warning message S6 in response to the statistical parameter S5 being greater than a preset threshold value, and may determine not to output the warning message S6 in response to the statistical parameter S5 being less than or equal to the preset threshold value. The calculation module 122 may transmit the warning message S6 to a terminal device of a relevant person (for example, the subject, the subject's family, the subject's caregiver or medical staffer, etc.) through the transmitter 130, to remind the person that the subject is at risk of neurological disorder.

In an embodiment, the calculation module 122 may determine whether to output the warning message S6 according to the correlation signal S4 and the statistical parameter S5. For example, the calculation module 122 may input the correlation signal S4 and the statistical parameter S5 to a pre-trained machine learning model, to allow the machine learning model to determine whether to output the warning message S6 according to the correlation signal S4 and the statistical parameter S5.

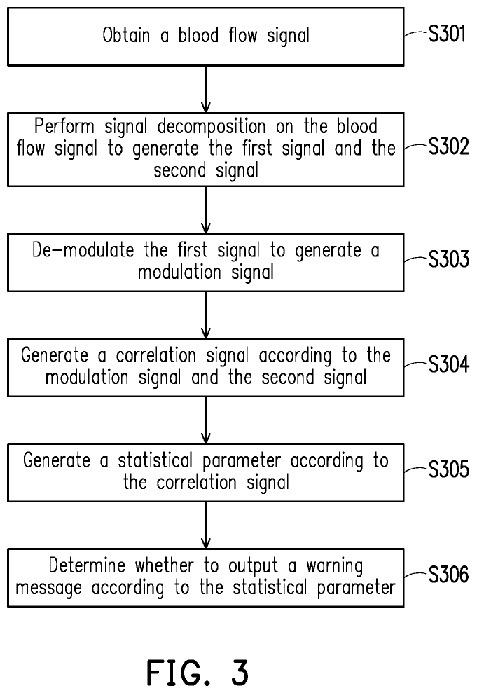

FIG. 3 illustrates a flow chart of a method for risk assessment of neurological disorder according to an embodiment of the disclosure. The method for risk assessment may be implemented by the electronic device 100 shown in FIG. 1. In step S301, a blood flow signal is obtained. In step S302, signal decomposition is performed on the blood flow signal to generate a first signal and a second signal. In step S303, the first signal is de-modulated to generate a modulation signal. In step S304, a correlation signal is generated according to the modulation signal and the second signal. In step S305, a statistical parameter is generated according to the correlation signal. In step S306, whether to output a warning message is determined according to the statistical parameter.

In summary, the electronic device of the disclosure may determine whether a subject is at risk of neurological disorder according to the subject's blood flow signal. Since the blood flow signal may be obtained by a non-intrusive measurement method, the subject does not need to endure the discomfort caused by an intrusive measurement method. In addition, the subject does not need to receive various types of clinical measurements. After obtaining the blood flow signal, the electronic device may perform signal decomposition on the blood flow signal to obtain two different signals, so as to use the two different signals to calculate the statistical parameter that may be used to determine whether the subject is at risk of neurological disorder. If the statistical parameter exceeds a preset range, the electronic device may output a warning message to notify a relevant person. For example, an electronic device may send a warning message to notify the subject to go to a hospital as soon as possible to be checked, so a subject with neurological disorder can be diagnosed and receive treatment in the early stage of neurological disorder.

What is claimed is:

1. An electronic device for risk assessment of neurological disorder, comprising:
a transceiver;
a storage medium, storing a plurality of modules; and
a processor, coupled to the storage medium and the transformer, accessing and performing the modules, wherein the modules comprise:
a data collection module, obtaining a blood flow signal through the transmitter; and
a calculation module, performing signal decomposition on the blood flow signal to generate a first signal and a second signal, de-modulating the first signal to generate a modulation signal, generating a correlation signal according to the modulation signal and the second signal, generating a statistical parameter according to the correlation signal, and determining whether to output a warning message through the transmitter according to the statistical parameter.

2. The electronic device according to claim 1, wherein the blood flow signal is a cerebral blood flow velocity signal, the first signal is a blood pulse signal, and the second signal is a trend signal.

3. The electronic device according to claim 2, wherein the calculation module performs the signal decomposition according to one of the following:
peak-valley interpolation, empirical mode decomposition, and de-trend fluctuation analysis.

4. The electronic device according to claim 2, wherein the calculation module performs the peak-valley interpolation on the blood flow signal to generate the first signal, and subtracts the first signal from the blood flow signal to generate the second signal.

5. The electronic device according to claim 2, wherein the calculation module performs the empirical mode decomposition on the blood flow signal to generate the first signal and the second signal, wherein the first signal is an intrinsic mode signal, and the second signal is a residue signal.

6. The electronic device according to claim 2, wherein the calculation module performs the de-trend fluctuation analysis on the blood flow signal to generate the first signal, and subtracts the first signal from the blood flow signal to generate the second signal.

7. The electronic device according to claim 1, wherein the statistical parameter at least comprises one of the following: a mean, a standard deviation, an interquartile range, and a coefficient of variation.

8. The electronic device according to claim 1, wherein the modulation signal corresponds to amplitude modulation.

9. The electronic device according to claim 1, wherein the calculation module determines whether to output the warning message according to the correlation signal and the statistical parameter.

10. A method for risk assessment of neurological disorder, comprising:
obtaining a blood flow signal;
performing signal decomposition on the blood flow signal to generate a first signal and a second signal;
de-modulating the first signal to generate a modulation signal;
generating a correlation signal according to the modulation signal and the second signal;
generating a statistical parameter according to the correlation signal; and
determining whether to output a warning message according to the statistical parameter.
